SEQUENCE LISTING (7 sequences)

SEQ ID NO: 1 - protein, 21 aa, Artificial Sequence
  synthetic peptide
ARTKQTARKSTGGKAPRKQLA

SEQ ID NO: 2 - protein, 19 aa, Artificial Sequence
  synthetic peptide
GGRGGFGGRGGFGGRGGFG

SEQ ID NO: 3 - protein, 40 aa, Artificial Sequence
  synthetic peptide
ARTKQTARKSTGGKAPRKQLATKAARKSAPATGGVKKPHR

SEQ ID NO: 4 - protein, 21 aa, Artificial Sequence
  synthetic peptide
LGKGGAKRHRKVLRDNIQGIT

SEQ ID NO: 5 - protein, 30 aa, Artificial Sequence
  synthetic peptide
ATKAARKSAPATGGVKKPHRYRPGTVALRE

SEQ ID NO: 6 - protein, 34 aa, Artificial Sequence
  synthetic peptide
GGSRAHSSHLKSKKGQSTSRHKKLMFKTEGPDSD

SEQ ID NO: 7 - protein, 350 aa, Artificial Sequence
  synthetic peptide
MDDQQALNSIMQDLAVLHKASRPALSLQETRKAKSSSPKKQNDVRVKFEHRGEKRILQFPRPVKLEDLRSKAKIAFGQSMDLHYTNNELVIPLTTQDDLDKAVELLDRSIHMKSLKILLVINGSTQATNLEPLPSLEDLDNTVFGAERKKRLSIIGPTSRDRSSPPPGYIPDELHQVARNGSFTSINSEGEFIPESMDQMLDPLSLSSPENSGSGSCPSLDSPLDGESYPKSRMPRAQSYPDNHQEFSDYDNPIFEKFGKGGTYPRRYHVSYHHQEYNDGRKTFPRARRTQGTSLRSPVSFSPTDHSLSTSSGSSIFTPEYDDSRIRRRGSDIDNPTLTVMDISPPSRSP

(19) United States
(12) Patent Application Publication
(10) Pub. No.: US 2016/0052955 A1
(43) Pub. Date: Feb. 25, 2016

(54) METHYLTRANSFERASE INHIBITORS FOR TREATING CANCERS

(71) Applicant: SLOAN-KETTERING INSTITUTE FOR CANCER RESEARCH, New York, NY (US)

(72) Inventors: Minkui LUO, New York, NY (US); Weihong ZHENG, New York, NY (US)

(73) Assignee: SLOAN-KETTERING INSTITUTE FOR CANCER RESEARCH, New York, NY (US)

(21) Appl. No.: 14/784,078

(22) PCT Filed: Apr. 15, 2014
(86) PCT No.: PCT/US2014/034118

§ 371 Date: Oct. 13, 2015

Related U.S. Application Data
(60) Provisional application No. 61/812,393, filed on Apr. 16, 2013.

(51) Int. Cl. C07H 19/16

(52) U.S. Cl. CPC C07H 19/16

(57) ABSTRACT

Compounds having methyltransferase inhibitory activity are disclosed. The compounds have the structure

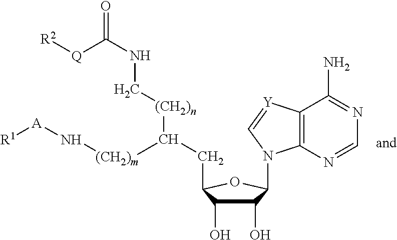

and are useful in the treatment of cancer and similar diseases associated with inappropriate methyltransferase activity.

### CROSS REFERENCE TO RELATED APPLICATIONS

This application claims priority from U.S. provisional application 61/812,393, filed Apr. 16, 2013, the entire disclosure of which is incorporated herein by reference.

### FIELD OF THE INVENTION

The invention relates to chemical compounds having methyltransferase inhibitory activity and their use in the treatment of diseases and conditions associated with inappropriate methyltransferase activity.

### BACKGROUND OF THE INVENTION

Epigenetics is inheritable information not encoded in DNA manifested through control of gene expression, thereby controlling a range of cellular activity, including determining cell fate, stem cell fate and regulating proliferation. Epigenetic control over gene expression is accomplished in at least four ways: (1) covalent histone modification, (2) covalent DNA modification, (3) histone variation, and (4) nucleosome structure and DNA/histone contact points. Epigenetic control through one mechanism can influence the other suggesting a combinatorial regulation, as evidenced by the methylation of histones being implicated in the modulation of DNA methylation.

Covalent histone modifications, a key mechanism involved in epigenetic control, include: (1) lysine acetylation, (2) lysine and arginine methylation, (3) serine and threonine phosphorylation, (4) ADP-ribosylation, (5) ubiquitination, and (6) SUMOylation. Specific enzymatic activities are associated with these modifications and in the case of histone methylation, methyltransferases catalyze the transfer of a methyl group from cofactor S-adenosylmethionine to a lysine or arginine, producing S-adenosylhomocysteine as a by-product. Methyltransferases can also modify residues in other cellular proteins, e.g. the tumor suppressor p53.

Histone methyltransferases fall into subgroups that include arginine methyltransferases, SET-domain containing methyltransferases SU(VAR)3-9, E(Z) and TRX, and DOT-like methyltransferase hDOT1L. Families of SET-domain containing methyltransferases have been identified and include SUV39, SET1, SET2 and RIZ.

The disruption of the normal functions of methyltransferases has been implicated in human diseases. Members of different classes of methyltransferases are implicated in cancer and representative examples for the subgroups and subclasses are provided: (1) hDOT1L, a member of the DOT-like methyltransferases, is linked to leukemogenesis [Nature Cell Biology, 8:1017-1028 (2006); Cell, 121:167-178 (2005); Cell, 112:771-723 (2003)]. (2) EZH2, a SET1 methyltransferase, is up-regulated in tumor cell lines and has been linked to breast, gastric and prostate cancers [British Journal of Cancer, 90:761-769 (2004)]. (3) SUV39-1/2, SUV39 methyltransferases, have been linked to signaling pathways regulating cancer cell growth and differentiation [Genetica, 117(2-3):149-58 (2003)]. (4) NSD1, a SET2 subclass methyltransferase, has been linked to acute myeloid leukemia and Sotos syndrome, a predisposition to cancer [Molecular Cell Biology, 24(12):5184-96 (2004)]. (5) EVI1, a RIZ methyltransferase, is overexpressed in solid tumors and leukemia [Proceeding of the National Academy of Sciences, 93:1642-1647 (1996)]. (6) Related enzymes, namely SMYD2, are lysine methyltransferases that modify the tumor suppressor protein, p53 and through this activity, may function as an oncogene that interferes with p53's protective functions [Nature, 444(7119):629-632 (2006)]. (7) SMYD3, a SET-domain containing lysine methyltransferase, is involved in cancer cell proliferation [Nature Cell Biology, 6(8):731-740 (2004)]. (8) CARM1 (also known as PRMT4), an arginine methlytransferase, is linked to prostate cancer [Prostate, 66(12):1292-301 (2006)], breast cancer [Wang et al., Cancer Cell 25, 21-36, (2014)] and to myeloid leukemia [Vu et al., Cell Reports 5, 1625-1638, (2013)].

Inappropriate methyltransferase activities thus represent attractive targets for therapeutic intervention by small molecule inhibitors. In fact, inhibitors of SUV(AR) histone methyltransferase [Nature Chemical Biology, 1:143-145 (2005)] and protein arginine methyltransferase [Journal of Biological Chemistry, 279:23892-23899 (2004)] have been described. The present invention relates to novel synthetic compounds effective as inhibitors of inappropriate histone methyltransferase activities. As a consequence of their inhibition of histone methyltransferase activity, these compounds would be useful in treating human diseases, such as cancer, particularly breast cancer, prostate cancer and hematological malignancies, such as leukemias and lymphomas, e.g. acute and chronic lymphoblastic and myelogenous leukemia, as well as Hodgkin's and non-Hodgkin's lymphomas.

### SUMMARY OF THE INVENTION

In one aspect, the invention relates to compounds of general formula I, which are potent and selective inhibitors of lysine and arginine methyltransferases:

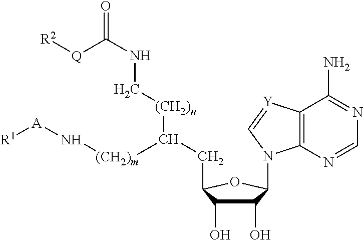

wherein:
Y is N or CH;
Q is NH or 0;
A is chosen from direct bond, (C₁-C₂o)hydrocarbon, (C₁-C₂₀)oxaalkyl and (C₁-C₂₀)azaalkyl;
R¹ is chosen from hydrogen, —C(═NH)NH₂, —C(═NH)NH(C₁-C₁₀)hydrocarbon, fluoro(C₁-C₆)hydrocarbon, and —CH(NH₂)COOH;
R² is R³, or when Q is NH, R² may additionally be —COR³ or —COOR³;
R³ is chosen from H, (C₁-C₂₀) hydrocarbon, substituted aryl, heteroaryl and substituted heteroaryl;
m is 0, 1 or 2;
and
n is 1,2 or 3.

In these compounds, A is a bivalent moiety and R¹ is a substituent on A. The members of this genus are effective as inhibitors of methyltransferase activities and therefore, are useful for the inhibition, prevention and suppression of various pathologies associated with such activities, such as, for example, cancer cell and cancer stem cell fate differentiation, and cancer cell proliferation and cell cycle regulation. The compounds are also useful research tools for studying protein methyl transferase biology.

In another aspect, the invention relates to pharmaceutical compositions comprising a therapeutically effective amount of at least one compound of general formula I and a pharmaceutically acceptable carrier.

In another aspect, the invention relates to a method for treating cancer comprising administering to a subject suffering from a cancer a therapeutically effective amount of a compound of formula I.

### DETAILED DESCRIPTION OF THE INVENTION

Throughout this specification the substituents are defined when introduced and retain their definitions.

In one aspect, the invention relates to compounds having general formula I:

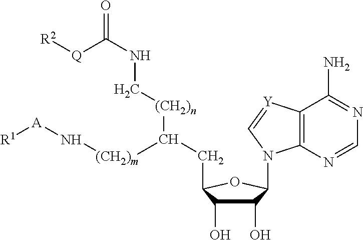

In some embodiments, R² is R³ and R³ is chosen from (C₁-C₆) alkyl and phenyl optionally substituted with one to three substituents chosen independently from halogen, haloalkyl, alkyl, acyl, hydroxyalkyl, hydroxy, alkoxy, haloalkoxy, oxaalkyl, carboxy, cyano, acetoxy, nitro, amino, alkylamino, dialkylamino, alkylthio, alkylsulfinyl, alkylsulfonyl, alkylsulfonylamino arylsulfonyl, arylsulfonylamino and benzyloxy. In some embodiments, R³ is chosen from (C₁-C₆) alkyl and para-monosubstituted phenyl.

In some embodiments n is 1. In some embodiments n is 3. In some embodiments n is 2. In some embodiments m is 0 or 1.

In some embodiments, Q is NH; in others Q is O.

In some embodiments, R¹-A is chosen from (C₁-C₆)alkyl, benzyl and (C₃-C₆)oxaalkyl. In these embodiments, R¹ is conceptually H and A is, for example, —(CH₂CH₂CH₂)—; or R¹ is conceptually H and A is

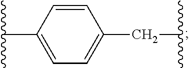 [SMILES: *Cc1ccc(*)cc1]

or R¹ is H and A is —(CH₂OCH₂CH₂CH₂)—. In other embodiments, R¹-A is chosen from hydrogen and —C(═NH)NH₂. In both these embodiments, A is a direct bond.

In all of the foregoing embodiments, Y may be CH, i.e. the heterocycle is 7-deazapurine (also known as 7H-pyrrolo[2,3-d]pyrimidine) or Y may be N, i.e. the heterocycle is purine.

For convenience and clarity certain terms employed in the specification, examples and claims are described herein.

Unless otherwise specified, alkyl (or alkylene) is intended to include linear or branched saturated hydrocarbon structures and combinations thereof. Alkyl refers to alkyl groups of from 1 to 20 carbon atoms, preferably 1 to 10 carbon atoms, more preferably 1 to 6 carbon atoms. Examples of alkyl groups include methyl, ethyl, propyl, isopropyl, n-butyl, s-butyl, t-butyl and the like. Lower alkyl refers to alkyl groups of from 1 to 4 carbon atoms.

Cycloalkyl is a subset of hydrocarbon and includes cyclic hydrocarbon groups of from 3 to 8 carbon atoms. Examples of cycloalkyl groups include c-propyl, c-butyl, c-pentyl, norbornyl and the like.

C₁ to C₂₀ hydrocarbon includes alkyl, cycloalkyl, polycycloalkyl, alkenyl, alkynyl, aryl and combinations thereof. Examples include benzyl, phenethyl, cyclohexylmethyl, adamantyl, camphoryl and naphthylethyl. Hydrocarbon refers to any substituent comprised of hydrogen and carbon as the only elemental constituents.

Unless otherwise specified, the term “carbocycle” is intended to include ring systems in which the ring atoms are all carbon but of any oxidation state. Thus (C₃-C₁₂) carbocycle refers to both non-aromatic and aromatic systems, including such systems as cyclopropane, benzene and cyclohexene. Carbocycle, if not otherwise limited, refers to monocycles, bicycles and polycycles. (C₈-C₁₂) Carbopolycycle refers to such systems as norbornane, decalin, indane and naphthalene

Alkoxy or alkoxyl refers to groups of from 1 to 20 carbon atoms, preferably 1 to 10 carbon atoms, more preferably 1 to 6 carbon atoms of a straight or branched configuration attached to the parent structure through an oxygen. Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 2002 edition, ¶196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples of azaalkyl include ethylaminoethyl and aminohexyl.

As used herein, the term “optionally substituted” may be used interchangeably with “unsubstituted or substituted”. The term “substituted” refers to the replacement of one or more hydrogen atoms in a specified group with a specified radical. For example, substituted alkyl, aryl, cycloalkyl, heterocyclyl etc. refer to alkyl, aryl, cycloalkyl, or heterocyclyl wherein one or more H atoms in each residue are replaced with halogen, haloalkyl, alkyl, acyl, alkoxyalkyl, hydroxyloweralkyl, carbonyl, phenyl, heteroaryl, benzenesulfonyl, hydroxy, loweralkoxy, haloalkoxy, oxaalkyl, carboxy, alkoxycarbonyl [—C(═O)O-alkyl], alkoxycarbonylamino [HNC(═O)O-alkyl], carboxamido [—C(═O)NH₂], alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkylsulfonyl, alkylsulfonylamino, arylsulfonyl, arylsulfonylamino, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. “Oxo” is also included among the substituents referred to in “optionally substituted”; it will be appreciated by persons of skill in the art that, because oxo is a divalent radical, there are circumstances in which it will not be appropriate as a substituent (e.g. on phenyl). In one embodiment, 1, 2 or 3 hydrogen atoms are replaced with a specified radical. In the case of alkyl and cycloalkyl, more than three hydrogen atoms can be replaced by fluorine; indeed, all available hydrogen atoms could be replaced by fluorine. Such compounds (e.g.perfluoroalkyl) fall within the class of “fluorohydrocarbons”. In preferred embodiments, substituents are halogen, haloalkyl, alkyl, acyl, hydroxyalkyl, hydroxy, alkoxy, haloalkoxy, oxaalkyl, carboxy, cyano, acetoxy, nitro, amino, alkylamino, dialkylamino, alkylthio, alkylsulfinyl, alkylsulfonyl, alkylsulfonylamino arylsulfonyl, arylsulfonylamino and benzyloxy.

Substituents Rⁿ are generally defined when introduced and retain that definition throughout the specification and in all independent claims.

As used herein, and as would be understood by the person of skill in the art, the recitation of “a compound”—unless expressly further limited—is intended to include salts of that compound. Thus, for example, the recitation “a compound of formula I” as depicted above, which incorporates a substituent COOH, would include salts in which the substituent is COO⁻M⁺, wherein M is any counterion. Similarly, formula I as depicted above depicts a substituent NH₂, and therefore would also include salts in which the substituent is NH₃⁺X⁻, wherein X is any counterion. Compounds containing a COOH substituent may commonly exist as zwitterions, which are effectively internal salts. In a particular embodiment, the term “compound of formula I” refers to the compound or a pharmaceutically acceptable salt thereof.

The term “pharmaceutically acceptable salt” refers to salts whose counter ion derives from pharmaceutically acceptable non-toxic acids and bases. Suitable pharmaceutically acceptable acids for salts of the compounds of the present invention include, for example, acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. Suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

It will be recognized that the compounds of this invention can exist in radiolabeled form, i.e., the compounds may contain one or more atoms containing an atomic mass or mass number different from the atomic mass or mass number usually found in nature. Alternatively, a plurality of molecules of a single structure may include at least one atom that occurs in an isotopic ratio that is different from the isotopic ratio found in nature. Radioisotopes of hydrogen, carbon, phosphorous, fluorine, chlorine and iodine include ²H, ³H, ¹¹C, ¹³C, ¹⁴C, ¹⁵N, ³⁵S, ¹⁸F, ³⁶Cl, ¹²⁵I, ¹²⁴I an ¹³¹I respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Compounds that contain isotopes ¹¹C, ¹³N, ¹⁵O, ¹²⁴I and ¹⁸F are well suited for positron emission tomography. Radiolabeled compounds of formula I of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Persons of skill will readily appreciate that compounds described herein, when appropriately labeled as described above, can be employed in a method of identifying (i.e. labeling) specific methyltransferase enzymes in the presence of other enzymes, including other methyltransferase enzymes, for which their affinity is lower. Usually two orders of magnitude difference in affinity will be sufficient to distinguish between enzymes. Using methods well known to persons of skill in the art, specific methyltransferase enzymes can be localized in tissues, cells and organelles. A further aspect of the invention described herein is thus a method of identifying and/or localizing specific methyltransferase enzymes.

Although this invention is susceptible to embodiment in many different forms, preferred embodiments of the invention are shown. It should be understood, however, that the present disclosure is to be considered as an exemplification of the principles of this invention and is not intended to limit the invention to the embodiments illustrated. It may be found upon examination that certain members of the claimed genus are not patentable to the inventors in this application. In this event, subsequent exclusions of species from the compass of applicants' claims are to be considered artifacts of patent prosecution and not reflective of the inventors' concept or description of their invention; the invention encompasses all of the members of the genus I that are not already in the possession of the public.

While it may be possible for the compounds of formula Ito be administered as the raw chemical, it is preferable to present them as a pharmaceutical composition. According to a further aspect, the present invention provides a pharmaceutical composition comprising a compound of formula I or a pharmaceutically acceptable salt or solvate thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof. The compositions may be formulated for oral, topical or parenteral administration. For example, they may be given intravenously, intraarterially, subcutaneously, and directly into the CNS—either intrathecally or intracerebroventricularly.

Formulations include those suitable for oral, parenteral (including subcutaneous, intradermal, intramuscular, intravenous and intraarticular), rectal and topical (including dermal, buccal, sublingual and intraocular) administration. The compounds are preferably administered orally or by injection (intravenous or subcutaneous). The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity. The formulations may conveniently be presented in unit dosage form and may be prepared by any of the methods well known in the art of pharmacy. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

It should be understood that in addition to the ingredients particularly mentioned above, the formulations of this invention may include other agents conventional in the art having regard to the type of formulation in question, for example those suitable for oral administration may include flavoring agents.

As used herein, “treatment” or “treating,” or “palliating” or “ameliorating” are used interchangeably herein. These terms refers to an approach for obtaining a therapeutic benefit in the form of eradication or amelioration of the underlying disorder being treated. Also, a therapeutic benefit is achieved with the eradication or amelioration of one or more of the physiological systems associated with the underlying disorder such that an improvement is observed in the patient, notwithstanding that the patient may still be afflicted with the underlying disorder. The compositions may be administered to a patient at risk of developing a particular disease, or to a patient reporting one or more of the physiological symptoms of a disease, even though a diagnosis of this disease may not have been made.

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes described herein, the person of ordinary skill can readily envision those groups that would be suitable as “protecting groups”. Suitable groups for that purpose are discussed in standard textbooks in the field of chemistry, such as Protective Groups in Organic Synthesis by T.W. Greene [John Wiley & Sons, New York, 1991], which is incorporated herein by reference.

A comprehensive list of abbreviations utilized by organic chemists appears in the first issue of each volume of the Journal of Organic Chemistry. The list, which is typically presented in a table entitled “Standard List of Abbreviations”, is incorporated herein by reference.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials are either commercially available, synthesized as described in the examples or may be obtained by the methods well known to persons of skill in the art. The synthetic methods parallel those described in PCT application WO2013/063417, the entire contents of which are incorporated herein by reference.

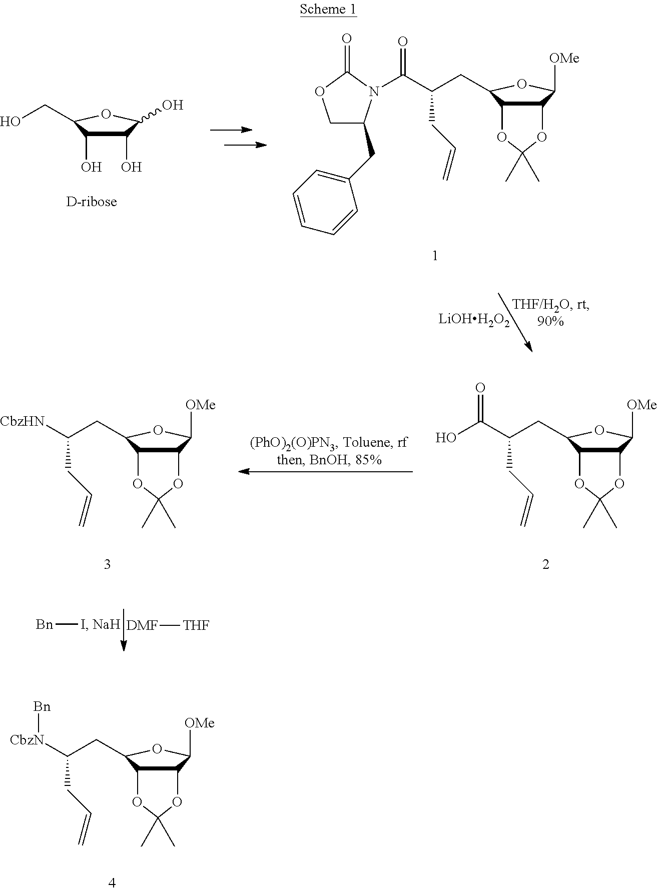

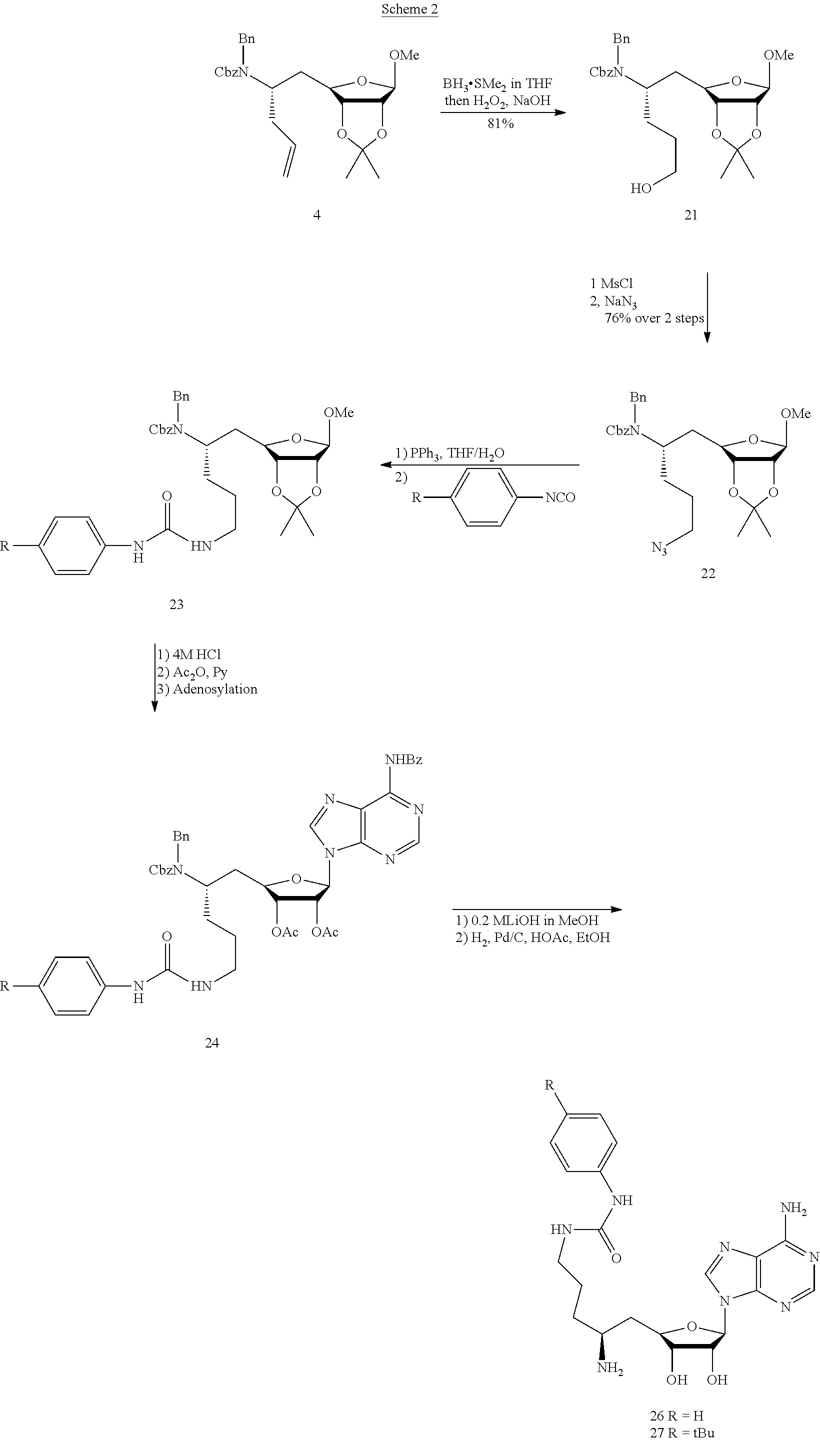

The synthesis of compounds 26 and 27 was started with intermediate 4. The hydroboration of compound 4, after oxidative work-up, gave primary alcohol. The conversion of alcohol into corresponding amino derivate was accomplished via azide substitution and Staudinger reduction. It was readily reacted with isocyanate to construct the urea. From this precursor, routine adenosylation and deprotection, which have been described in PCT application U.S. Ser. No. 12/62157, delivered target compounds.

Azide 22 ¹H NMR (CDCl3, 600 MHz): δ1.11-1.22(m, 5H), 1.33-1.34(m, 0.5H), 1.39-1.42(m, 3.5H), 1.50-1.59(m, 3H), 1.74-1.78(m, 0.5H), 1.95-1.98(m, 1.5H), 2.80(s, 1H), 2.93(t, 1H, J=6.6 Hz), 3.17(s, 1.3H), 3.27(s, 1.7H), 4.11(dd, 0.6H, J=10.8 Hz, 3.3 Hz), 4.15(d, 0.6H, J=15.4 Hz), 4.23(d, 0.4H, J=5.6 Hz), 4.39(d, 0.6H, J=5.8 Hz), 4.44(d, 0.4H, J=5.8 Hz), 4.51(d, 0.4H, J=5.8 Hz), 4.60-4.62(m, 0.6H), 4.83(s, 0.4H), 4.89(s, 0.6H), 5.11-5.18(m, 2H), 7.16-7.32(m, 10H); ¹³C NMR (CDCl3, 600 MHz rotamers): δ 24.87, 24.93, 25.29, 25.82, 25.96, 26.45, 29.73, 30.26, 31.07, 31.61, 34.68, 37.61, 38.69, 50.03, 50.59, 50.84, 50.91, 54.59, 54.93, 55.24, 55.41, 67.18, 67.64, 83.82, 83.92, 84.21, 84.33, 85.47, 85.53, 109.99, 110.18, 112.24, 112.31, 127.49, 127.97, 127.02, 128.14, 128.26, 128.38, 128.51, 128.54, 136.52, 138.52, 138.73, 155.70, 157.30. MS(ESI)m/z: 547 ([M+Na]⁺; HRMS: calculated for C₂₈H₃₆N₄O₆Na ([M+Na]⁺) 547.2533, found 547.2518.

Compound 26: MS(ESI)m/z: 457([M+H]⁺; HRMS: calculated for C₂₁H₂₉N₈O₄ ([M+H]⁺) 457.2312, found 457.2316.

Compound 27: ¹H NMR (MeOD, 500 MHz): δ 1.21(s, 9H), 1.54-1.57(m, 2H), 1.64-1.69(m, 2H), 2.01-2.06(m, 1H), 2.11-2.16(m, 1H), 3.12-.315(m, 2H), 3.37-3.41(m, 1H), 4.11-4.16(m, 1H), 4.20(t, 1H, J=5.8 Hz), 5.58(dd, 1H, J=5.4HZ, 4 Hz), 5.97(d, 1H, J=3.8 Hz), 7.16(d, 2H, J=6.8 Hz), 7.21(d, 2H, J=5.8 Hz), 8.34(s, 1H), 8.35(s, 1H); MS(ESI)m/z: 513([M+H]⁺; HRMS: calculated for C₂₅H₃₇N₈O₄ ([M+H]⁺) 513.2938, found 513.2925.

Other compounds in which m is zero and n is two can be made in analogous fashion by substituting the appropriate isocyanate in the conversion of 22 to 23 in Scheme 2.

Compounds in which m is one and n is 1 can be made as shown in Scheme 3 below:

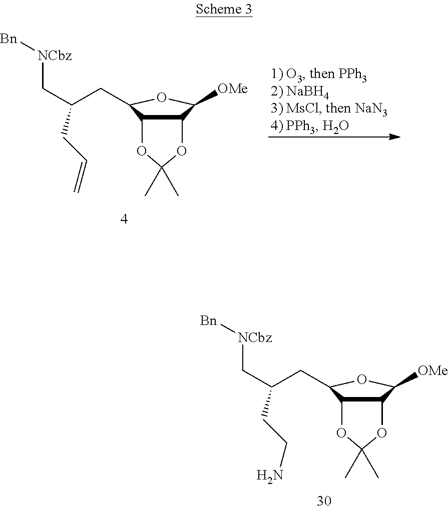

Compounds in which m is one and n is 3 can be made as shown in Scheme 4 below:

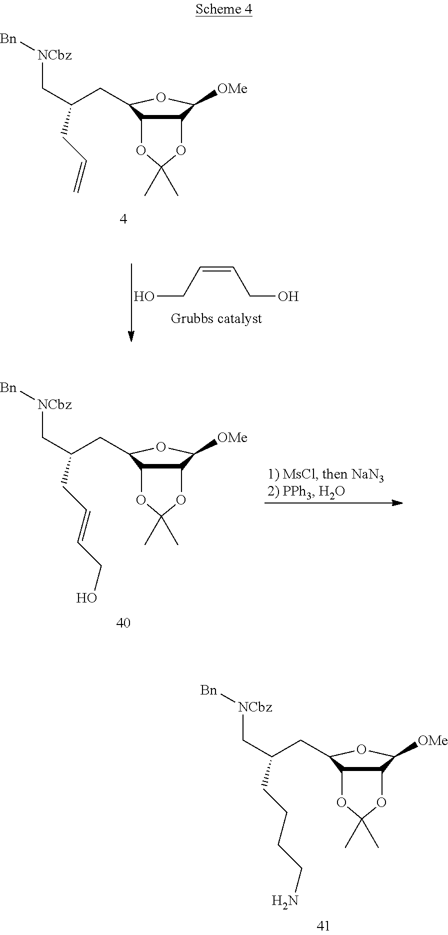

Compounds in which m is two can be made as shown in Scheme 5 below:

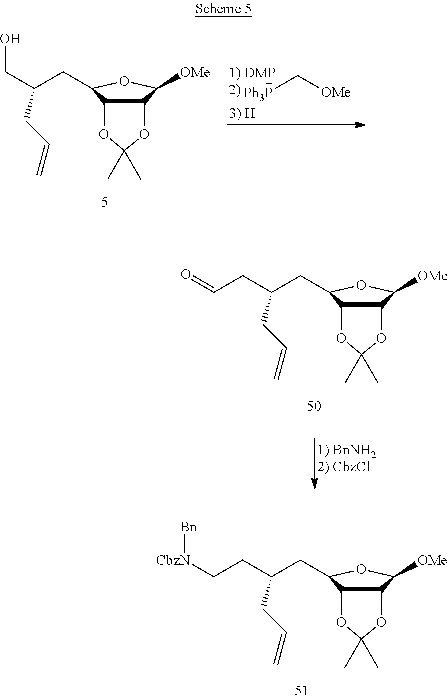

Compounds in which Y is CH may be synthesized by using the appropriate deazapurine as shown in Scheme 6:

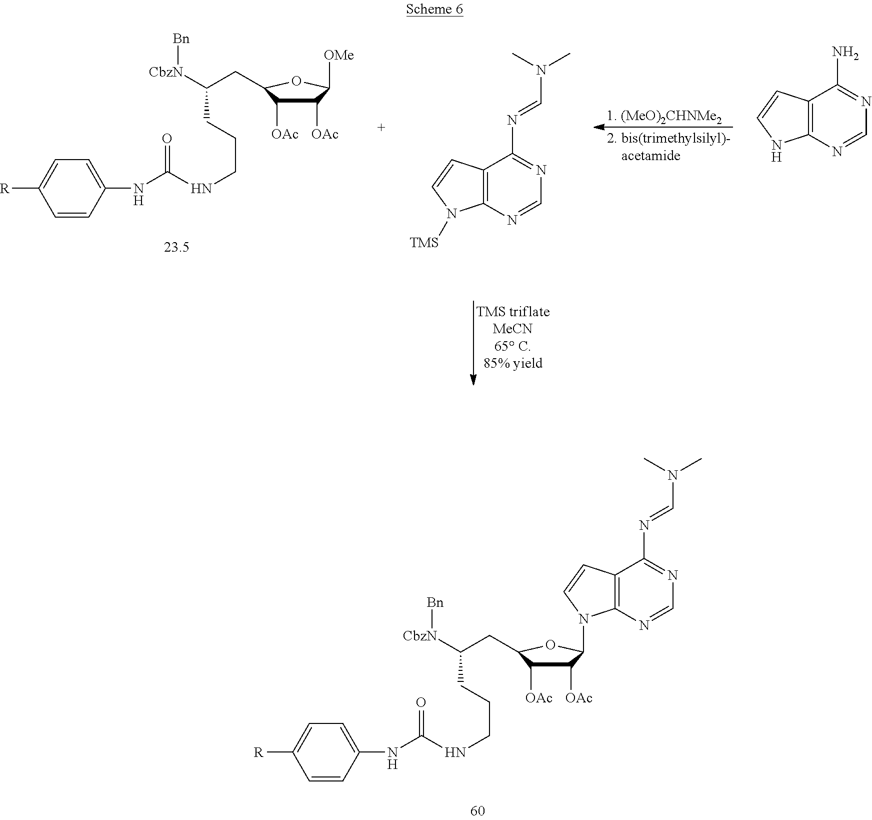

Compounds in which m is one and n is 2 can be made as shown in Scheme 7 below:

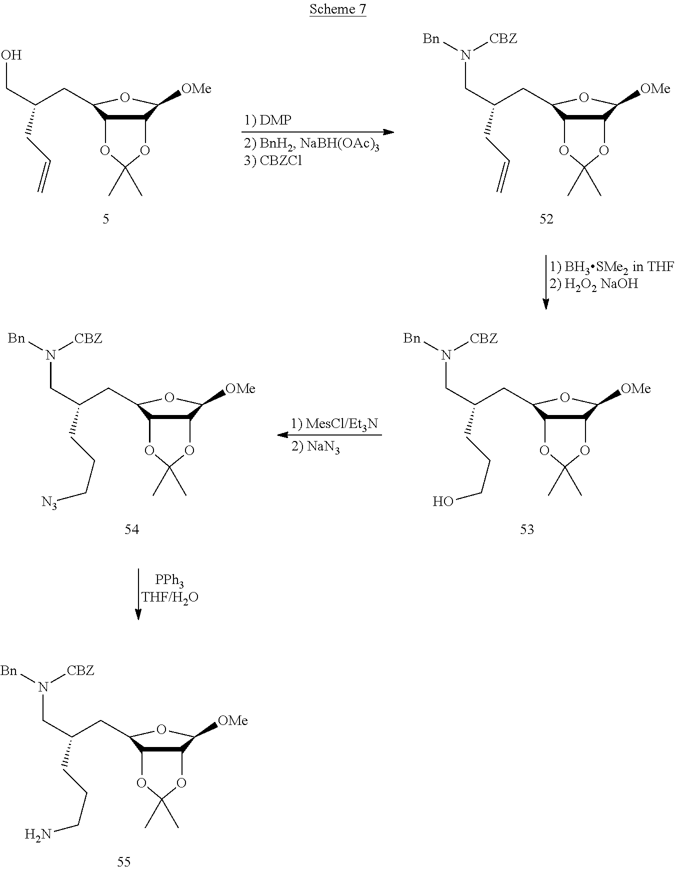

Two particular examples, 28 and 29, were made from intermediate 55 as shown:

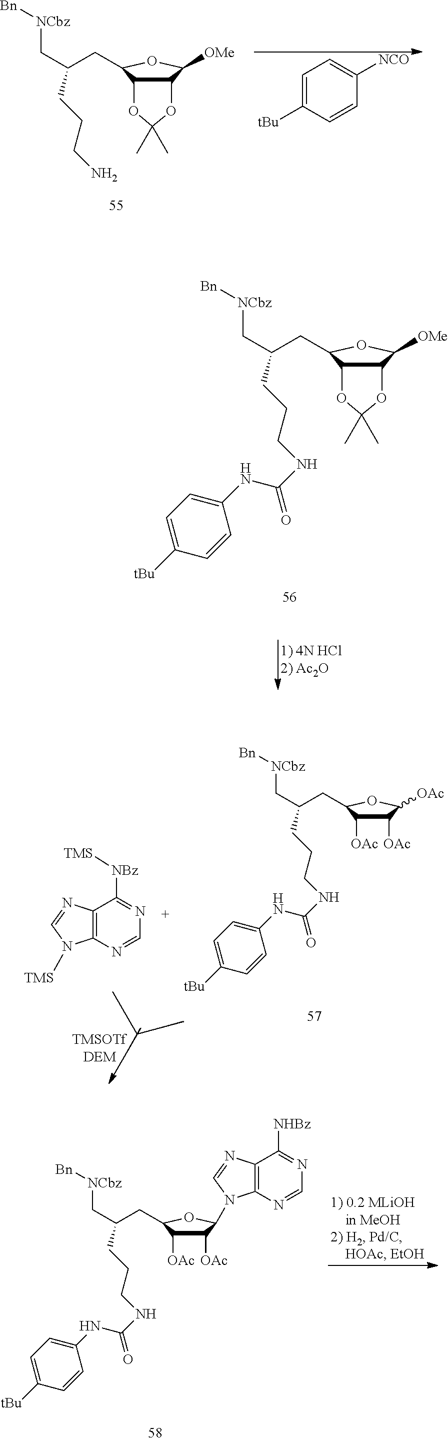

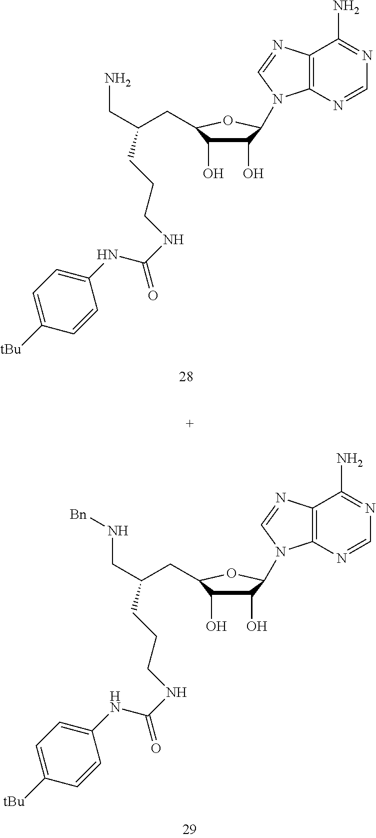

Example 28: ¹H NMR (MeOD, 500 MHz): δ 1.29(s, 9H), 1.40-1.65(m, 4H), 1.82-1.90(m, 1H), 1.95-2.05(m, 1H), 2.87 (dd, 1H, J=12.5, 7.5), 3.01 (dd, 1H, J=12.5, 7.5 Hz), 3.12-0.325(m, 3H), 4.11-4.16(m, 1H), 4.18(t, 1H, J=5.5 Hz), 4.72 (t, 1H, J=5.5 Hz), 5.97(d, 1H, J=3.8 Hz), 7.16(d, 2H, J=8.7 Hz), 7.21(d, 2H, J=8.7 Hz), 8.30(s, 1H), 8.32(s, 1H).

Example 29:¹H NMR (MeOD, 500 MHz): δ 1.29(s, 9H), 1.42-1.63(m, 4H), 1.85-1.93(m, 1H), 2.02-2.15(m, 2H), 3.02 (dd, 1H, J=12.5, 7.5), 3.05-3.18 (m, 3H), 3.19-3.25(m, 1H), 4.08-4.12(m, 3H), 4.19(t, 1H, J=5.8 Hz), 4.65 (t, 1H, J=5.8 Hz), 5.96(d, 1H, J=3.8 Hz), 7.20-7.40(m, 10H), 8.26(s, 1H), 8.31(s, 1H); HRMS: calculated for C₃₃H₄₅N₈O₄ ([M+H]⁺) 617.3564, found 617.3549.

The compounds described above were tested as described below:

Methylation Reaction. The 20 μL methylation reaction was carried out at ambient temperature using two mixtures: A. 10 μl of enzyme mixture in the assay buffer containing 50 mM Hepes (pH=8.0), 0.005% Tween-20, 5 μg/ml BSA and 1 mM TCEP; B. 10 μl of a mixture of 1.5 04, 0.15 μCi [³H-Me]-SAM cofactor and 3 μM of the corresponding peptide substrate in the same assay buffer. After A and B were mixed for a designated time period, the reaction mixture was examined with our filter-paper assay.

Conditions for the enzymes:

| | [Enzyme mixture] | [Enzyme]_{final} | | Reaction Time |
|---|---|---|---|---|
| Enzyme | (nM) | (nM) | Substrate | (h) |
| G9a (913-1913) | 40 | 20 | H3 (1-21 aa) | 1 |
| GLP1 (951-1235) | 20 | 10 | H3 (1-21 aa) | 1 |
| SUV39H2 (112-410) | 10 | 5 | H3 (1-21 aa) | 4 |
| SET7/9 Full-length | 300 | 150 | H3 (1-21 aa) | 3 |
| PRMT1 (10-352) | 200 | 100 | RGG | 1.5 |
| PRMT3 (211-531) | 200 | 100 | RGG | 3 |
| CARM1 (19-608) | 600 | 300 | H3 (1-40 aa) | 7 |
| SET8 (191-352) | 2000 | 1000 | H4 (10-30 aa) | 8 |
| SETD2 (1347-1711) | 500 | 250 | H3 (20-50 aa) | 4 |
| SMYD2 Full-length | 100 | 50 | p53 (360-393 aa) | 10 |
| SMYD3 | 250 | 125 | MAP3K2 (1-350 aa) | 2 |
| SETDB1Full-length | 15 | 7.5 | H3 (1-21 aa) | 15 |
| DOT1L | 100 | 50 | Nucleosomes | 6 |

H3 (1-21-aa):
(SEQ ID NO: 1)
ARTKQTARKSTGGKAPRKQLA
RGG:
(SEQ ID NO: 2)
GGRGGFGGRGGFGGRGGFG
H3 (1-40 aa):
(SEQ ID NO: 3)
ARTKQTARKSTGGKAPRKQLATKAARKSAPATGGVKKPHR
H4 (10-30 aa):
(SEQ ID NO: 4)
LGKGGAKRHRKVLRDNIQGIT
H3 (20-50 aa):
(SEQ ID NO: 5)
ATKAARKSAPATGGVKKPHRYRPGTVALRE
p53 (360-393 aa):
(SEQ ID NO: 6)
GGSRAHSSHLKSKKGQSTSRHKKLMFKTEGPDSD
MAP3K2 (1-350 aa):
(SEQ ID NO: 7)
MDDQQALNSIMQDLAVLHKASRPALSLQETRKAKSSSPKKQNDVRVKFE
HRGEKRILQFPRPVKLEDLRSKAKIAFGQSMDLHYTNNELVIPLTTQDD
LDKAVELLDRSIHMKSLKILLVINGSTQATNLEPLPSLEDLDNTVFGAE
RKKRLSIIGPTSRDRSSPPPGYIPDELHQVARNGSFTSINSEGEFIPES
MDQMLDPLSLSSPENSGSGSCPSLDSPLDGESYPKSRMPRAQSYPDNHQ
EFSDYDNPIFEKFGKGGTYPRRYHVSYHHQEYNDGRKTFPRARRTQGTS
LRSPVSFSPTDHSLSTSSGSSIFTPEYDDSRIRRRGSDIDNPTLTVMDI
SPPSRSP

Filter-paper Assay. This assay relies on Whatman P-81 filter paper, which binds peptides but not SAM. Protein Methyl Transferases (PMTs) transfer ³H-Me of [³H-Me]-SAM to peptide substrates and the resultant ³H-methylated, filter-paper-bound peptide is quantified with a scintillation counter. Briefly, 6 μl of the methylation reaction was spotted onto Whatman P-81 phosphocellulose filter paper (1.2×1.2 cm²) to immobilize the ³H-labeled peptide. After drying in air for 20 min, the filter paper was immersed into 20 mL of 50 mM Na₂CO₃/NaHCO₃ buffer (pH=9.2), and washed 5 times for 10 min each time. The washed filter paper was then transferred to a 20 ml scintillation vial containing 1 mL of distilled water and 10 mL of Ultima Gold scintillation cocktail or 7 mL scintillation vial containing 0.5 mL od distilled water and 5 mL of scintillation cocktail (PerkinElmer). The radioactivity was quantified by a Beckman LS6000IC liquid scintillation counter.

Dose-response Curves and IC50. Twice the PMT concentration was incubated for 10 min with varied concentration of inhibitors (0.1-400 μM stocks), into which 10 μl of the PMT peptide substrate and radioactive cofactor (3 μM of the corresponding peptide and 1.5 μM, 0.15 μCi [³H-Me]-SAM) were added. After incubating the reaction mixture for the respective reaction time, the conversion was quantified with the filter paper assay as described above. The inhibition was expressed as the percentage between the high control (no inhibition) and the low control (no enzyme) as follows: Percentage Inhibition=[(high control−reading)/(high control−low control)]×100%. Each experiment was performed in triplicate. The IC₅₀ values were obtained by fitting inhibition percentage versus inhibitor concentration using GraphPad Prism5 software.

The results are shown in the following table, in which S-adenosyl homocysteine (SAH) and sinefungin (SIN) are controls:

| | | | Exam- | Exam- | Exam- | Exam- |
|---|---|---|---|---|---|---|
| | SAH | Sinefungin | ple 26 | ple 27 | ple 28 | ple 29 |
| G9a | 6.66 | 18.86 | >100 | >100 | >100 | >100 |
| GLP1 | 5.03 | 32.02 | >100 | >100 | >100 | >100 |
| SET7/9 | >100 | 1.14 | >100 | >100 | >100 | >100 |
| SET8 | >100 | >100 | >100 | >100 | >100 | >100 |
| SETD2 | 2.94 | 28.44 | 42.7 | 23.4 | >100 | >100 |
| PRMT1 | 8.59 | 1.034 | ~100 | >100 | >100 | >100 |
| (100 nm, | | | | | | |
| RGG) | | | | | | |
| PRMT3 | 39.5 | 28.17 | 5.9 | 35.4 | >50 | >50 |
| SUV39H2 | 0.63 | 4.58 | >100 | >100 | >100 | ~10 |
| CARM1 | 1.90 | 0.44 | 1.9 | 0.1 | 0.73 | <0.2 |
| SMYD2-FL | ~50 | 0.22 | 61.6 | ~100 | >100 | >100 |
| SMYD3 | | | | | >100 | >100 |
| SETDB1-FL | 0.95 | 8 | 55 | ~100 | | |
| DOT1L | 2.2 | 53.4 | 0.84 | 0.14 | 0.07 | 0.17 |

Compounds 27, 28 and 29 showed remarkable inhibitory activity to CARM1 and to DOT1L. Compared to the positive control, the replacement of aminoacid with phenyl urea surprisingly did not have a deleterious effect on the affinity to some enzymes. Given that this replacement reduced the overall polarity of sinefungin, it is predicted that compounds of the invention will exhibit increased membrane permeability.

Compounds that show selective inhibition of one or a few families of PMTs are of greater interest as candidates for use in therapy, since it is believed that broad spectrum inhibition is likely to be associated with a higher probability of side effects. In this regard, compounds 27, 28 and 29 are of interest.

1. A compound of formula I 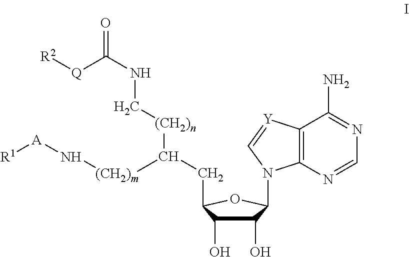
wherein:
Y is N or CH;
Q is NH or O;
A is chosen from direct bond, (C₁-C₂₀)hydrocarbon, (C₁-C₂₀)oxaalkyl and (C₁-C₂₀)azaalkyl;
R¹ is chosen from hydrogen, —C(═NH)NH₂, —C(═NH)NH(C₁-C₁₀)hydrocarbon, fluoro(C₁-C₆) hydrocarbon, and —CH(NH₂)COOH;
R² is R³, or when Q is NH, R² may additionally be —COR³ or —COOR³;
R³ is chosen from H, (C₁-C₂₀) hydrocarbon, substituted aryl, heteroaryl and substituted heteroaryl; m is 0, 1 or 2; and
n is 1, 2 or 3.

2. A compound according to claim 1 wherein R³ is chosen from (C₁-C₆) alkyl and phenyl optionally substituted with one to three substituents chosen independently from halogen, haloalkyl, alkyl, acyl, hydroxyalkyl, hydroxy, alkoxy, haloalkoxy, oxaalkyl, carboxy, cyano, acetoxy, nitro, amino, alkylamino, dialkylamino, alkylthio, alkylsulfinyl, alkylsulfonyl, alkylsulfonylamino, arylsulfonyl, arylsulfonylamino and benzyloxy.

3. A compound according to claim 2 wherein R³ is chosen from (C₁-C₆) alkyl and para-monosubstituted phenyl.

4. A compound according to claim 1 wherein n is 1.

5. A compound according to claim 1 wherein n is 3.

6. A compound according to claim 1 wherein n is 2.

7. A compound according to claim 6 wherein m is 0 or 1.

8. A compound according to claim 1 wherein Q is NH.

9. A compound according to claim 8 wherein R² is chosen from (C₁-C₆) alkyl, (C₃-C₇) cycloalkyl and phenyl substituted with one to three substituents chosen independently from halogen, haloalkyl, alkyl, acyl, hydroxyalkyl, hydroxy, alkoxy, and haloalkoxy.

10. A compound according to claim 9 wherein R² is phenyl substituted with halogen, halo(C₁-C₆)alkyl, (C₁-C₆) alkyl, acyl, hydroxymethyl, hydroxy, (C₁-C₆) alkoxy, and halo(C₁-C₆)alkoxy; n is 2; m is 0 or 1; and R¹-A- is hydrogen or (C₁-C₂₀) hydrocarbon.

11. A compound according to claim 1 wherein Q is O.

12. A compound according to claim 1 wherein R¹-A is chosen from hydrogen, benzyl and —C(═NH)NH₂.

13. A compound according to claim 1 wherein Y is CH.

14. A compound according to claim 1 wherein Y is N.

15. A pharmaceutical composition comprising a pharmaceutically acceptable carrier and a compound according to claim 1.

16. A method for inhibiting the activity of a methyltransferase enzyme comprising bringing said methyltransferase enzyme into contact with a compound according to claim 1.

17. A method for selectively inhibiting the activity of a first methyltransferase enzyme in the presence of a second methyltransferase enzyme comprising bringing both of said methyltransferase enzymes into contact with a compound according to claim 1.

18. A method of treating cancer in a patient suffering from cancer comprising administering to said patient a therapeutically effective amount of a compound according to claim 1.

19. A method according to claim 18 wherein said cancer is breast cancer or prostate cancer.

20. A method according to claim 18 wherein said cancer is a hematological malignancy.

21. A method according to claim 20 wherein said cancer is leukemia.
